(19) United States
(12) Patent Application Publication
(10) Pub. No.: US 2003/0065040 A1
(43) Pub. Date: Apr. 3, 2003

(54) Pharmaceutical compositions including sampatrilat dispersed in a lipoidic vehicle

(76) Inventors: Amir H. Shojaei, Gaithersburg, MD (US); Rong-Kun Chang, Rockville, MD (US); Beth A. Burnside, Bethesda, MD (US)

Correspondence Address: CARELLA, BYRNE, BAIN, GILFILLAN,, CECCHI, STEWART & OLSTEIN, 6 Becker Farm Road, Roseland, NJ, 07068, US

(21) Appl. No.: 10/061,804

(22) Filed: Feb. 1, 2002

Related U.S. Application Data
(63) Continuation-in-part of application No. 09/773,838, filed on Feb. 1, 2001.

(51) Int. Cl. A61K 31/40

(52) U.S. Cl. 514/786

(57) ABSTRACT

A pharmaceutical composition comprising a dispersion in which an inhibitor of angiotensin converting enzyme and neutral endopeptidase, such as sampatrilat, is dispersed in a lipoidic vehicle. Such a composition has improved systemic bioavailability.

This application is a continuation-in-part of application Ser. No. 09/773,838, filed Feb. 1, 2001.

### FIELD OF THE INVENTION

This invention relates to pharmaceutical compositions including inhibitors of angiotensin converting enzyme and/or neutral endopeptidase, which have improved systemic bioavailability. More particularly, this invention relates to pharmaceutical compositions containing sampatrilat, dispersed in a lipoidic vehicle.

### BACKGROUND OF THE INVENTION

Sampatrilat is a dual inhibitor of angiotensin converting enzyme (ACE) and neutral endopeptidase (NEP), with potential application as an antihypertensive agent as well as a treatment for congestive heart failure. Because of this unique dual modality, sampatrilat does not cause a sudden and significant drop in blood pressure after administration of the first dose and has a much lower propensity to cause common side effects such as dry cough. The oral bioavailability of sampatrilat has been reported to be as low as 5% in dogs and 2% in man. Clinical pharmacokinetic data show generally low but persistent plasma drug exposure following single and multiple doses.

### SUMMARY OF THE INVENTION

In accordance with an aspect of the present invention, there is provided a pharmaceutical composition comprising a dispersion. The dispersion comprises an agent selected from the group consisting of inhibitors of angiotensin converting enzyme and inhibitors of neutral endopeptidase, dispersed in a lipoidic vehicle.

In accordance with the present invention, there is provided a lipoidic pharmaceutical composition comprising a dispersion, said dispersion comprising an agent selected from the group consisting of inhibitors of angiotensin converting enzymes and inhibitors of neutral endopeptidase.

In accordance with the present invention, there is provided a pharmaceutical composition comprising a dispersion, said dispersion comprising an agent selected from the group consisting of inhibitors of angiotensin converting enzymes and inhibitors of neutral endopeptidase, dispersed in a lipoidic vehicle.

In accordance with the present invention there is provided a method for the treatment or prevention of cardiovascular disorders including hypertension and heart failure comprising the step of administering a pharmaceutically effective amount of a formulation of the present invention to a subject in need of such treatment or prevention.

In accordance with the present invention there is provided the use of a pharmaceutically effective amount of a formulation of the present invention for treating or preventing cardiovascular disorders including hypertension and heart failure.

In accordance with the present invention there is provided a method for the manufacture of a formulation comprising the steps of dispersing an agent selected from the group consisting of inhibitors of angiotensin converting enzymes and inhibitors of neutral endopeptidase, in a lipoidic vehicle.

### BRIEF DESCRIPTION OF THE DRAWINGS

FIG. 1 shows the Sampatrilat Plasma concentration versus time profiles for the formulations described in example 2;

FIG. 2 shows the Sampatrilat Plasma concentration versus time profiles for the formulations described in example 3; and

FIG. 3 shows the plasma concentration of sampatrilat in the single dose study (SPD442.101).

### DETAILED DESCRIPTION OF THE INVENTION

In one embodiment, the formulations of the present invention comprise those wherein the following embodiments are present, either independently or in combination.

Inhibitors of angiotensin converting enzyme and/or neutral endopeptidase which may be included in the composition include, but are not limited to, sampatrilat, fasidotril, omapatrilat, enalaprilat, and mixtures thereof.

In a further embodiment, inhibitors of angiotensin converting enzyme and/or neutral endopeptidase which may be included in the composition include, but are not limited to, sampatrilat, omapatrilat, enalaprilat, and mixtures thereof.

In one embodiment, the inhibitor of angiotensin converting enzyme and/or neutral endopeptidase is sampatrilat.

The pharmaceutical agent is present in the composition in an amount of from about 0.5 wt. % to about 25 wt. % preferably from about 1 wt. % to about 14 wt. %.

The lipoidic vehicle, in one embodiment, is present in the composition in an amount of from about 40 wt. % to about 99 wt. %, preferably from about 86 wt. % to about 99 wt. %.

In one embodiment, the lipoidic vehicle is a glyceride and derivatives thereof. Preferably, the glyceride is selected from the group consisting of medium chain glycerides and caprylocaproyl macrogolglycerides, and mixtures thereof.

In one embodiment, the glyceride is a medium chain glyceride. Medium chain glycerides which may be employed in the composition of the present invention include, but are not limited to, medium chain monoglycerides, medium chain diglycerides, caprylic/capric triglyceride, glyceryl monolaurate, caprylic/capric glycerides, glycerylmonocaprylate, glyceryl monodicaprylate, caprylic/capric linoleic triglyceride, and caprylic/capric/succinic triglyceride.

In another embodiment, the glyceride is a caprylocaproyl macrogolglyceride. Caprylocaproyl macrogolglycerides which may be employed include, but are not limited to, polyethylene glycosylated glycerides, or PEGylated glycerides. PEGylaed glycerides which may be employed in the composition include, but are not limited to, mixtures of monoglycerides, diglycerides, and triglycerides and monoesters and diesters of polyethylene glycol, polyethylene glycosylated almond glycerides, polyethylene glycosylated corn glycerides, and polyethylene glycosylated caprylic/capric triglyceride.

In one embodiment, the composition further comprises a sorbent, which sorbs the liquid dispersion of the agent dispersed in the lipoidic vehicle and solidifies the liquid dispersion and converts the liquid dispersion to a free-flowing powder. The sorbent may be present in the composition in an amount of from about 20 wt. % to about 60 wt. %, preferably from about 45 wt. % to about 55 wt. %. Sorbents which may be employed include, but are not limited to, dicalcium phosphate, silicon dioxide, magnesium oxide, magnesium aluminometasilicate, microcrystalline cellulose, and maltodextrin. In one embodiment, the sorbent is dicalcium phosphate.

The composition also may include wetting agents, surfactants (e.g., sorbitan monooleate, sorbitan monolaurate, polysorbate, etc.), cosurfactants (e.g., cetyl alcohol, glyceryl monostearate, sodium carboxy methyl cellulose, cetyl trimethylammonium bromide, and lauryl dimetbylbenzylammonium chloride), thickening agents (e.g., silicon dioxide, glyceryl behenate, etc.), adsorbents (e.g., silicon dioxide, maltodextrin, granulated calcium phosphate, etc.), and processing aids such as lubricants, glidants, and antiadherants.

The particles of the agent, such as sampatrilat, do not have to be dissolved partially or fully in the lipoidic medium in order to have enchanced bioavailability. The agent, such as sampatrilat, in a lipoidic medium is a coarse dispersion, and is analogous to a pharmaceutical suspension in terms of particle size and physical behavior.

In addition, the dispersions of the present invention do not require or include a water phase or a specific geometric orientation or particle size. The particles of the agent, such as sampatrilat, merely are dispersed in the medium, which consists of a homogeneous oleaginous phase. Microparticulate or nanoparticulate sampatrilat drug particles are not required for enhanced bioavailability.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLE 1

### Preparation and In Vitro Drug Release Characterization of Sampatrilat Formulations

In this example, liquid filled capsules were prepared by placing all ingredients in a suitable container, and the ingredients were homogenized at high speed for 4 minutes. The liquid dispersion then was encapsulated using appropriately sized hard gelatin capsules. The capsules then were sealed using a hydroalcoholic solution of gelatin.

Powder filled capsules or directly compressed tablets were formed by placing all ingredients, except the adsorbent powder, in a suitable container. The mixture then was homogenized for 4 minutes at high speed. An appropriate amount of adsorbent powder then was added, and the mixture was triturated until a free flowing solid dispersion was obtained. The solid dispersion then was encapsulated using appropriately sized hard gelatin capsules or the dispersion was formulated into tablets by direct compression.

The formulations are given in Table 1 below.

TABLE 1

| | PD0058-24B | | PD0058-33 | | PD0058-36 | | PD0058-15 | | PD0058-34 | |
|---|---|---|---|---|---|---|---|---|---|---|
| Ingredients | 1 | 2 | 1 | 2 | 1 | 2 | 1 | 2 | 1 | 2 |
| Sampatrilat | 10 | 1.4 | 10 | 0.72 | 10 | 1.4 | 10 | 5.0 | 10 | 5.0 |
| Labrasol ® | 345 | 49.3 | 345 | 26.64 | 303 | 43.3 | — | — | — | — |
| Capmul | 345 | 49.3 | 345 | 24.64 | 303 | 43.3 | — | — | — | — |
| MCM ® | | | | | | | | | | |
| Span 80 ® | — | — | — | — | 42 | 6.0 | — | — | — | — |
| Deionized | — | — | — | — | 42 | 6.0 | — | — | — | — |
| Water | | | | | | | | | | |
| Fujicalin | — | — | 700 | 50.0 | — | — | — | — | 190 | 95.0 |
| SG ® | | | | | | | | | | |
| Lactose | — | — | — | — | — | — | 190 | 95.0 | — | — |

1 = composition in mg per capsule
2 = composition in % weight
Note:
PD0058-15 and PD0058-34 contain no enhancers in the formulations. These two formulations serve as control.
Labrasol ® is a trade name for caprylocaproyl macrogolglyceride and is marketed by Gattefosse Corp.
Capmul MCM ® is a trade name for medium chain mono- and diglycerides and marketed by Abitec Corp.
Span 80 ® is a trade name for sorbitan monooleate and marketed by ICI Chemical.
Fujicalin SG ® is a trade name for dicalcium phosphate and marketed by Fuji Chemical.

A Vankel dissolution tester (Van Kel Industries, Edison, N.J.) was used for all dissolution studies. The apparatus was calibrated according to USP23. The dissolution in 0.1N hydrochloric acid (pH 1.2) or deionized water was tested using the paddle method (USP Apparatus II), employing 900 ml of dissolution medium at a temperature of 37° C. and an agitation rate of 50 rpm. Samples at specific time points, i.e., 15, 30, 45, 60 min., were removed and filtered through a 10 μm filter. The filtered samples were kept in screw cap glass test tubes until analysis. An HPLC system comprised of an autosampler and a pump and a UV detector was used for sample analysis. 50 μl of the dissolution samples were injected directly on the HPLC C18 column using a mixture of pH 7.0 phosphate buffer acetonitrile (92:8) as the mobile phase. The dissolution data are given in Table 2 below.

TABLE 2
Dissolution Data for Sampatrilat Capsule Formulations

| | |
|---|---|
| Time | PD0058-32A |

| (min) | 1 | 2 |
|---|---|---|
| 15 | 99.0 ± 5.1 | 101.0 ± 5.9 |
| 30 | 105.0 ± 6.0 | 107.0 ± 7.5 |
| 45 | 106.0 ± 7.8 | 112.0 ± 6.1 |
| 60 | 107.0 ± 7.6 | 115.0 ± 5.7 |

1 = percent dissolved using deionized water as the dissolution medium
2 = percent dissolved using 0.1 N HCl as the dissolution medium
Note:
The data represent the mean percent dissolved ± standard deviation of three replicates. PD0058-32A had the same composition as that for PD0058-36

Content uniformity tests were conducted by determining the amount of sampatrilat in each of 10 samples A through J) using a high pressure liquid chromatography (HPLC) methodology specific for sampatrilat detection. The relative standard deviation (RSD) of the average of the 10 capsules is then taken as an indicator of content uniformity with % RSD<5.0 as passing. The content uniformity data are given in Table 3 below.

TABLE 3
Content Uniformity Data for Sampatrilat Capsule Formulations

PD0058-32A

| Sample | 1 | 2 |
|---|---|---|
| A | 10.37 | 103.7 |
| B | 10.49 | 104.9 |
| C | 10.97 | 109.7 |
| D | 11.13 | 111.3 |
| E | 10.84 | 108.4 |
| F | 11.08 | 110.8 |
| G | 11.27 | 112.7 |
| H | 11.16 | 111.6 |
| I | 11.46 | 114.6 |
| J | 11.27 | 112.7 |
| Mean | 11.00 | 110.0 |

| | |
|---|---|
| % RSD | 3.16 |

1 = weight (mg) per capsule
2 = percent label claim per capsule
Note:
PD0058-32A had the same composition as that for PD0058-36

### EXAMPLE 2

### Preparation and in vivo Evaluation of Sampatrilat Formulations in Accordance with the Invention

Formulations tested were delivered in dogs as liquid-filled hard gelatin capsule dosage forms with 10 mg sampatrilat. Three formulations containing the enhancers as well as a control formulation were tested in vivo as part of the first dog study (n=6). Table 4 below provides a summary of the formulations prepared. The detailed procedural description is given below.

TABLE 4
Summary of Sampatrilat Formulations

| | | | Average | |
|---|---|---|---|---|
| | | Average | Total | |
| | | Weight of | Content | |
| | | components | Weight | Lot |
| Formulation | Composition | (mg/capsule) | (mg/capsule) | Number |
| Control | Lactose | 190 | 198 | PD0058-15 |
| | Sampatrilat | 10 | | |
| Formulation | Labrasol | 690 | 709 | PD0058-18 |
| #1 | Sampatrilat | 10 | | |
| Formulation | Labrasol | 345 | 706 | PD0058-24B |
| #2 | CapmulMCM | 345 | | |
| | Sampatrilat | 10 | | |
| Formulation | Capmul MCM | 690 | 703 | PD0058-26 |
| #3 | Sampatrilat | 10 | | |

The control formulation was prepared by using lactose as the diluent/filler. Pre-weighed amounts of sampatrilat (100 mg) and lactose (1900 mg) were triturated and mixed using a mortar and pestle. Appropriate amounts of this powder blend were encapsulated in size 00 Swedish orange hard gelatin capsules by hand filling. The filled capsules were then sealed with a hydroalcoholic solution of gelatin. The content weight of each capsule was 201, 196, 197, 196, 196, 195, 202, and 200 mg (PD0058-15).

For formulation #1, Labrasol was used as the vehicle and a liquid dispersion was prepared. Using a mortar and pestle, a total of 120 mg of sampatrilat was homogeneously dispersed in 8280 mg of Labrasol. Encapsulation was done by hand filling this dispersion into size 00 Swedish orange hard gelatin capsules. The filled capsules were then sealed with a hydroalcoholic solution of gelatin. The content weight of each capsule was 712, 714, 700, 707, 703, 714, 715, and 709 mg (PD0058-18).

Because formulation #2 contained Labrasol as well as Capmul MCM in equal proportions, a mixture (PD0058-24A) containing 5 g labrasol and 5 g Capmul MCM was first prepared. Using a mortar and pestle, 120 mg of sampatrilat was homogeneously dispersed in 8280 mg of the Labrasol/Capmul MCM mixture. Encapsulation was done by hand filling this dispersion into size 00 Swedish orange hard gelatin capsules. The filled capsules were then sealed with a hydroalcoholic solution of gelatin. The content weight of each capsule was 709, 714, 709, 701, 696, 715, 698, and 706 mg (PD0058-24B).

Formulation #3 was prepared by using a mortar and pestle to homogeneously disperse 120 mg of sampatrilat in 8280 mg of Capmul MCM. Encapsulation was done by hand filling this dispersion into size 00 Swedish orange hard gelatin capsules. The filled capsules were then sealed with a hydroalcoholic solution of gelatin. The content weight of each capsule was 712, 709, 701, 697, 704, 694, 693, and 712 mg (PD0058-26).

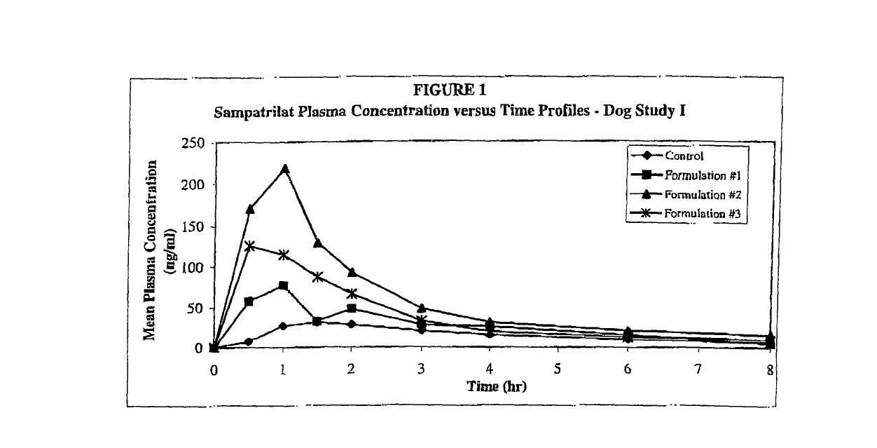

The average plasma concentration versus time profiles for all formulations studied are shown in FIG. 1 and the data summarized in Table 5. The mean Cₘₐₓ for control, formulation #1, formulation #2, and formulation #3 were 39, 164, 243 and 152 ng/ml, respectively. The mean AUC₀₋₄₈ for control, Formulation #1, Formulation #2, and Formulation #3 were 132, 987, 595 and 457, respectively. Though all the formulations were effective in increasing the Cₘₐₓ and AUC₀₋₄₈ compared to the control, a significantly lower inter-subject variation was achieved with Formulation #2 (Table 5).

Using the sampatrilat intravenous injection data obtained from a canine model in a previous study, the absolute oral bioavailability of sampatrilat was calculated for all formulations (Table 6). Due to the atypical plasma concentration vs. time profiles for two of the dogs in the formulation #1 study, the bioavailability calculations were performed after exclusion of this atypical data. Formulation #2 resulted in a significantly (P<0.05) greater bioavailability as compared to the control.

All three formulations tested in vivo resulted in enhanced sampatrilat concentrations and extent of drug absorption as compared to the control formulation. Statistically significant differences (P<0.05) were observed between formulation #2 and the control formulation. Formulation #2 resulted in a 5-fold increase in the oral bioavailability of sampatrilat as compared to the control.

TABLE 5
Mean Plasma Concentration of Sampatrilat from
All Formulations in Dogs (n = 6)

| Time (hr) | Control | Formulation #1 | Formulation #2 | Formulation #3 |
|---|---|---|---|---|
| | Mean Plasma Concentration (ng/mL) | | | |
| 0 | 0.00 | 0.00 | 0.00 | 0.00 |
| 0.5 | 6.15 | 93.42 | 170.94 | 125.66 |
| 1 | 29.23 | 64.23 | 218.77 | 114.44 |
| 1.5 | 33.46 | 27.49 | 129.24 | 87.89 |
| 2 | 30.18 | 44.94 | 92.80 | 66.33 |
| 3 | 20.46 | 29.74 | 48.24 | 32.75 |
| 4 | 12.77 | 31.30 | 31.28 | 19.75 |
| 6 | 8.15 | 30.02 | 19.74 | 12.48 |
| 8 | 2.10 | 29.98 | 14.59 | 9.59 |
| 24 | 0.00 | 22.72 | 0.00 | 3.50 |
| 48 | 0.00 | 0.00 | 0.00 | 0.00 |

Mean PK Parameters

| | | | | |
|---|---|---|---|---|
| Cₘₐₓ | 39.06 | 163.90 | 243.19 | 151.94 |
| Tₘₐₓ | 1.33 | 2.08 | 0.833 | 1.17 |
| AUC₀₋₄₈ | 132 | 987 | 595 | 457 |

TABLE 6
Absolute Oral Bioavailability of Sampatrilat
by Cross Study Comparison

| | Absolute Oral |
|---|---|
| Formulation | Bioavailability* (% ± SE) |
| Control | 7.6 ± 1.9 |
| Formulation #1 | 19.9 ± 4.4** |
| Formulation #2 | 35.3 ± 4.3 |
| Formulation #3 | 27.8 ± 8.9 |

*Results adjusted based on individual dog weights.
**Two dogs were excluded because of invalid results.

### EXAMPLE 3

### Effect of Additives on Sampatrilat Formulations

As part of the second dog study, three iterations of formulation #2 (from example 2) from the first dog study were tested in vivo in dogs along with one control formulation. The first formulation consisted of Fujicalin SG as an absorbent to solidify the lipoidic vehicle and the formulation as in the form of powder-filled capsule (PD0058-33). The second formulation consisted of Labrasol®, Capmul MCM®, and Span 80® (sorbitan monooleate), as a viscosity enhancing agent (PD0058-36). The third formulation was similar in composition and it differed in the ratio of drug to enhancers, where the amount of enhancers was reduced by 50% (PD0058-37). Table 7 provides the composition of all formulations tested in the second dog study (n=6).

The control formulation was prepared by using Fujicalin SG as the diluent/filler. Pre-weighed amounts of sampatrilat (97.35 mg) and Fujicalin SG (1915.89 mg) were triturated and mixed using a mortar and pestle. Appropriate amounts of this powder blend were encapsulated in size 00 Swedish orange hard gelatin capsules by hand filling. The filled capsules were then sealed with a hydroalcoholic solution of gelatin. The content weight of each capsule was 193, 195, 196, 201, 197, 195, 197, and 199 mg (PD0058-34).

TABLE 7
Summary of Sampatrilat Formulations

| | PD0058-33 | | PD0058-36 | | PD0058-37 | | | |
|---|---|---|---|---|---|---|---|---|
| | Formulation | | Formulation | | Formulation | | PD0058-34 | |
| | #2A | | #2B | | #2C | | Control | |
| Ingredients | 1 | 2 | 1 | 2 | 1 | 2 | 1 | 2 |
| Sampatrilat | 10 | 0.72 | 10 | 1.4 | 10 | 2.8 | 10 | 5.0 |
| Labrasol | 345 | 24.64 | 303 | 43.3 | 151.5 | 42.6 | — | — |
| Capmul | 345 | 24.64 | 303 | 43.3 | 151.5 | 42.6 | — | — |
| MCM | | | | | | | | |
| Span 80 | — | — | 42 | 6.0 | 21 | 6.0 | — | — |
| Deionized | — | — | 42 | 6.0 | 21 | 6.0 | — | — |
| Water | | | | | | | | |
| Fujicalin SG | 700 | 50.0 | — | — | | | 190 | 95.0 |

1 = composition in mg per capsule
2 = composition in % weight

For formulation #2A, 127.99 mg sampatrilat, 4139.79 mg Labrasol, and 4146.80 mg Capmul MCM were added and the mixture was homogenized for 4 minutes to a complete dispersion. To this dispersion 8.4735 g of Fujicalin SG was added by geometric dilution Fujicalin SG adsorbs the dispersion to form a powdered mixture. Encapsulation was done by hand filling this solid dispersion into size 000 hard gelatin capsules. The content weight of each capsule was 1408, 1409, 1398, 1401, 1404, 1397, 1407, and 1416 mg (PD0058-33).

Formulation #2B contained Labrasol, Capmul MCM, Span 80, and water, therefore to 122.19 mg sampatrilat, 3637.45 mg Labrasol, 3648.32 mg Capmul MCM, 524.29 mg Span 80, and 524.54 mg water were added and placed in a 50 ml glass beaker. The mixture was homogenized for 4 minutes to a complete dispersion. Encapsulation was done by hand filling this dispersion into size 00 Swedish orange hard gelatin capsules. The filled capsules were then sealed with a hydroalcoholic solution of gelatin. The content weight of each capsule was 708, 723, 728, 717, 705, 710, 717, and 726 mg (PD0058-36).

Formulation #2C was prepared similar to formulation #2, to 120.88 mg sampatrilat, 1837.29 mg Labrasol, 1823.68 mg Capmul MCM, 265.78 mg Span 80, and 244.85 mg water were added and placed in a 50 ml glass beaker. The mixture was homogenized for 4 minutes to a complete dispersion. Encapsulation was done by hand filling this dispersion into size 00 Swedish orange hard gelatin capsules. The filled capsules were then sealed with a hydroalcoholic solution of gelatin. The content weight of each capsule was 352, 357, 352, 358, 344, 358, 358, and 353 mg (PD0058-37).

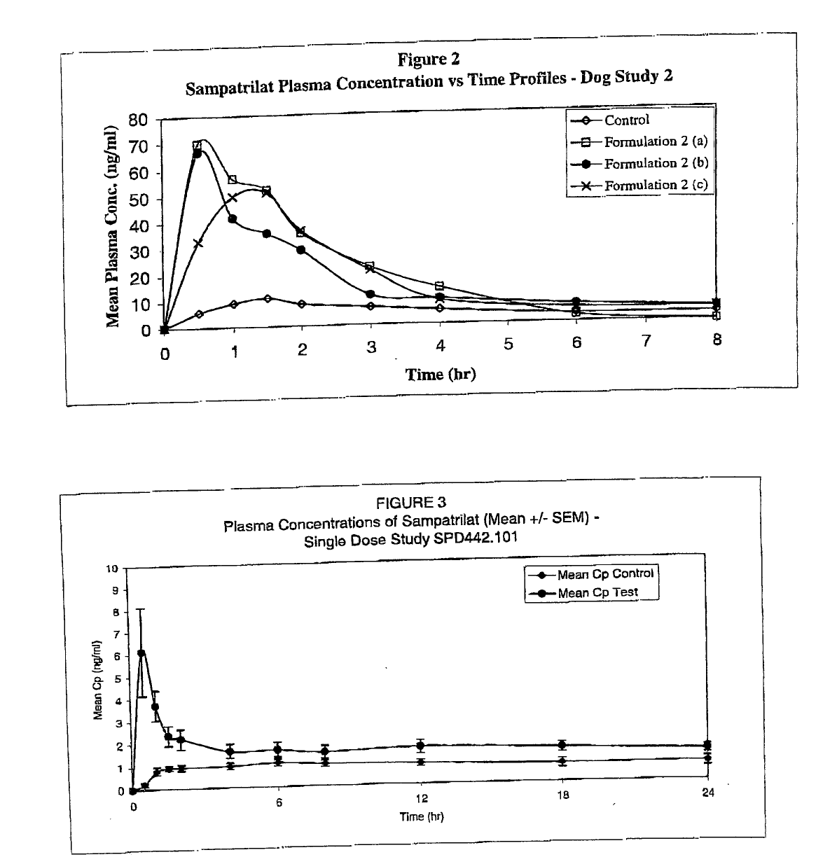

FIG. 2 along with Tables 8, 9 and 10 provide a summary of the second sampatrilat dog study (n=6).

TABLE 8
Mean Pharmacokinetic (PK) Parameters

| | | Formulation | Formulation | Formulation |
|---|---|---|---|---|
| PK Parameter | Control | #2A | #2B | #2C |
| Tₘₐₓ(hr) | 1.25 | 0.83 | 1.17 | 1.00 |
| Cₘₐₓ(ng/ml) | 17.16 | 80.57 | 69.83 | 55.74 |
| AUC₀₋₂₄(ng. hr/ml) | 98.00 | 163.83 | 387.50 | 199.20 |
| Ratio Cₘₐₓ | 1.00 | 4.58 | 3.97 | 3.17 |
| Ratio AUC₀₋₂₄ | 1.00 | 1.67 | 3.95 | 2.03 |

TABLE 8
Absolute Oral Bioavailability of Sampatrilat
by Cross Study Comparison

| | Absolute Oral Bioavailability* |
|---|---|
| Formulation | (% ± SE) |
| Control | 5.3 ± 2.7 |
| Formulation #2A | 8.6 ± 3.2 |
| Formulation #2B | 20.9 ± 5.9 |
| Formulation #2C | 10.7 ± 6.4 |

*Results adjusted based on individual dog weights.

TABLE 10
Enhancement Ratio Comparison Between
Dog Study I and Dog Study II

| | |
|---|---|
| | Enhancement Ratio |
| Formulation | (AUC_{formulation}/AUC_{control}) |

Dog Study I (n = 6)

| | |
|---|---|
| Control | 1.0 |
| Formulation #1 | 2.6 |
| Formulation #2 | 4.6* |
| Formulation #3 | 3.6 |

Dog Study II (n = 6)

| | |
|---|---|
| Control | 1.0 |
| Formulation #2A | 1.6 |
| Formulation #2B | 3.9* |
| Formulation #2C | 2.0 |

*Significant different (P < 0.05) from their respective control formulations

Even though all three formulations resulted in enhanced systemic absorption, a more physically stable outside of the capsule form of the original Formulation #2 (from dog study-1, example 2) yielded a low inter-subject variability along with a significant enhancement in systemic bioavailability (when comparing the formulations to their respective control formulations). However, the enhancement seen with original formulation #2 was superior to the one seen with formulation #2B, therefore the addition of water appears to play a negative role on the bioavailibility enhancement. The addition of water also creates unfavorable stability inside gelatin based or other capsules shells, where dissolution failure as well as capsule shell softening occurs.

### EXAMPLE 4

### Human Bioavailability Comparison Study of Sampatrilat Formulations

Sampatrilat formulations were tested in 16 volunteers in a single dose double blind crossover study. The composition of the formulations is listed in Table 11.

TABLE 11
Composition of sampatrilat formulations tested
in a human bioavailability comparison study

| | 01-0213 | 2008.00.002 |
|---|---|---|
| | (mg/capsule) | (mg/capsule) |
| | Reference | Test |
| Component | Formulation | Formulation |
| Sampatrilat | 50.00 | 50.00 |
| Caprylocaproyl | — | 257.00 |
| macrogolglycerides, EP | | |
| (Labrasol) | | |
| Glyceril caprylate/caprate | — | 257.00 |
| (Capmul MCM) | | |
| Sorbitan monooleate, | — | 30.00 |
| NF (Span 80) | | |
| Silicon dioxide, | — | 6.00 |
| USP (Cab-o-sil) | | |
| Dicalcium phosphate anhydrous, | 502.30 | — |
| granular, USP | | |
| Pregelatinized starch, NF | 81.30 | — |
| Magnesium stearate, USP | 6.40 | |
| Hard gelatin capsule, size 0EL | * | — |
| Hard gelatin capsule, size 00 | — | * |
| TOTAL | 640.00 | 600.00 |

*Excluded from weight calculations.

The average plasma concentration of sampatrilat for both reference (control) and test formulations are shown in FIG. 3 and table 12 along with the respective mean pharmacokinetic parameters. The test formulation was shown to be significantly (P<0.05) better than the reference formulation by providing for a 1.8 fold improvement in the extent of drug absorption and a 4.5 fold enhancement in the rate of drug absorption.

TABLE 12
Average Pharmacokinetic Parameters

| | Average Pharmacokinetic Parameters | | | | |
|---|---|---|---|---|---|
| | Cₘₐₓ | | AUC₀₋₃₆ | | |
| | (ng/ | Tₘₐₓ | (ng.hr/ | Enhancement | Enhancement |
| Formulation | ml) | (hr) | ml) | Ratio (Cmax) | Ratio (AUC) |
| Control | 1.42 | 14.0 | 31.4 | 1.00 | 1.00 |
| Formulation | | | | | |
| Test | 6.35 | 3.28 | 55.33 | 4.47 | 1.76 |
| Formulation | | | | | |

It is to be understood, however, that the scope of the present invention is not to be limited to the specific embodiments described above. The invention may be practiced other than as particularly described and still be within the scope of the accompanying claims.

What is claimed is:

1. A pharmaceutical composition comprising a dispersion, said dispersion comprising an agent selected from the group consisting of inhibitors of angiotensin converting enzymes and inhibitors of neutral endopeptidase, dispersed in a lipoidic vehicle.

2. The composition of claim 1 wherein said agent is sampatrilat.

3. The composition of claim 1 wherein said lipoidic vehicle is a glyceride.

4. The composition of claim 3 wherein said glyceride is selected from the group consisting of medium chain glycerides and caprylocaproyl macrogolglycerides, and mixtures thereof.

5. The composition of claim 4 wherein said glyceride is a medium chain glyceride.

6. The composition of claim 5 wherein said medium chain glyceride is selected from the group consisting of medium chain monoglycerides, medium chain diglycerides, caprylic/capric triglyceride, glyceryl monolaurate, caprylic/capric glycerides, glycerylmonocaprylate, glyceryl monodicaprylate, caprylic/capric linoleic triglyceride, and caprylic/capric/succinic triglyceride.

7. The composition of claim 4 wherein said glyceride is a caprylocaproyl macrogolglyceride.

8. The composition of claim 7 wherein said caprylocaproyl macrogolglyceride is a polyethylene glycosylated glyceride.

9. The composition of claim 8 wherein said polyethylene glycosylated glyceride is selected from the group consisting of mixtures of monoglycerides, diglycerides, and triglycerides and monoesters and diesters of polyethylene glycol, polyethylene glycosylated almond glycerides, polythylene glycosylated corn glycerides, and polyethylene glycosylated caprylic/capric triglyceride.

10. The composition of claim 1, and further comprising a sorbent.

11. The composition of claim 10 wherein said sorbent is dicalcium phosphate.

12. A method for the treatment or prevention of cardiovascular disorders including hypertension and heart failure comprising the step of administering a pharmaceutically effective amount of a formulation as defined in claim 1 to a subject in need of such treatment or prevention.

13. The use of a pharmaceutically effective amount of a formulation as defined in claim 1 for treating or preventing cardiovascular disorders including hypertension and heart failure.

14. A method for the manufacture of a formulation comprising the steps of dispersing an agent selected from the group consisting of inhibitors of angiotensin converting enzymes and inhibitors of neutral endopeptidase, in a lipoidic vehicle.
